(19) United States
(12) Patent Application Publication
(10) Pub. No.: US 2023/0052103 A1
(43) Pub. Date: Feb. 16, 2023

(54) METHOD OF FITTING A KNEE PROSTHESIS WITH ASSISTANCE OF AN AUGMENTED REALITY SYSTEM

(71) Applicant: PIXEE MEDICAL, Besançon (FR)

(72) Inventors: Romain Fissette, Besançon (FR); Sébastien Henry, La Crau (FR); Anthony Agustinos, Devecey (FR)

(73) Assignee: PIXEE MEDICAL, Besançon (FR)

(21) Appl. No.: 17/880,265

(22) Filed: Aug. 3, 2022

Related U.S. Application Data
(60) Provisional application No. 63/229,322, filed on Aug. 4, 2021.

(51) Int. Cl. A61F 2/46; A61B 34/20; A61B 90/00

(52) U.S. Cl. CPC A61F 2/461; A61B 34/20; A61B 90/37; A61B 90/361; A61B 2090/365; A61B 2034/252

(57) ABSTRACT

The method of fitting a knee prosthesis in a knee of a patient includes displaying, by a visual display device of a mobile augmented reality navigation system worn or carried by a user performing or assisting at least one navigation-assisted stage of the method, at least one visual element superposed on a view of at least a portion of a surgical scene of the fitting of the knee prosthesis. The visual element can be a 3D model of at least one portion of the knee of the patient, a 3D model of another component of the surgical scene, information relative to the at least one portion of the knee of the patient, and/or information relative to the other component of the surgical scene.

### RELATED APPLICATION

This application claims priority of provisional application No. 63/229,322 filed Aug. 4, 2021, whose content is hereby incorporated by reference herein in its entirety.

### FIELD OF THE INVENTION

The invention relates to protocols for fitting a knee prosthesis assisted by an augmented reality navigation system.

### BACKGROUND ART

Knee replacement surgery including partial or total knee replacement is an effective method to allow a patient's continued motion of the knee. The success of such a surgery highly depends on the precision of the position and orientation of the prosthetic components on the patient's bone structures. The surgery can be performed with a standard surgical technique involving regular metallic instruments provided by the implant manufacturer, or more recently with the assistance of a navigation station, involving digital equipment and dedicated surgical instruments, that can assist the surgeon in planning the surgery and performing it with precision.

### SUMMARY OF THE INVENTION

In one aspect of the present invention, a method is proposed to bring assistance to the surgeon, by providing simulation and/or information to the surgeon via an augmented reality system, for precisely fitting a knee prosthesis on a patient's knee joint.

In another aspect, an augmented reality navigation system provides simulation and/or assistance information relative to a navigated surgical instrument or instruments, so as to assist a surgeon in using these navigated instruments, notably positioning and/or orienting, at one or more stages of the knee prosthesis operation, or throughout the knee prosthesis operation. In another aspect, information on a navigated surgical instrument or instruments is acquired by a perception sensor of an augmented reality navigation system, and shown on a display of an augmented reality display device of the system. A perception device can integrate one or more sensors of various technologies, especially visual sensors, more particularly optical sensors, such as RGB camera, infrared camera, monochrome camera, stereovision system by passive or active camera, LiDAR, RADAR, ultrasound devices, etc. A single sensor can be used, or a plurality of sensors using the same or different technologies.

In another aspect, successive acquisitions by a perception sensor of relative positions of and/or orientations of a specific navigated surgical instrument or instruments allow successive displaying by the visual display of position and/or orientation and/or additional simulation and assistance information relative to the positioning of an object, such as an operating instrument or device, for example, a registration pointer and/or a ligament tensor and/or a component of a prosthesis. The acquired data includes, for example, measurements of joint spaces under tension by a ligament tensor, or orientation parameters of a bone cutting guide with respect to the patient's anatomy.

In another aspect, a visual display device of a mobile augmented reality navigation system, worn or carried by a user performing or assisting at least one, advantageously, at least two or more than two, navigation-assisted stages of the method, displays at least one visual element superposed on a view of at least a portion of a surgical scene of the fitting of the knee prosthesis. The at least one visual element can be a 3D model of at least one portion of the knee of the patient, a 3D model of another component of the surgical scene, information relative to the at least one portion of the knee of the patient, and/or information relative to the other component of the surgical scene. Non-limitatively, components of the surgical scene can include landmarks of the patient's knee, femur, or tibia; markers; instruments or portions of instruments, and/or a knee prosthesis or portions of a knee prosthesis.

In another aspect, a navigation-assisted stage of the method includes performing a manual action by the user or by a person assisted by the user, wherein the user or the person initiates the performance of the manual action, and modifies the performance of the manual action taking into account the at least one visual element. For example, a surgical gesture or the positioning of an instrument can be initiated, then adjusted in real time by a surgeon wearing an augmented reality device, taking into account the visual elements indicating a correct gesture or placement superposed onto the view of the surgical scene.

In another aspect, the visual display device can display simulation and/or assistance information relative to successive positioning, for example. Such successive visual elements can be stored in a memory included in or connected to the navigation system for simultaneous or later display. At least one perception sensor is advantageously included in or connected to the navigation system.

In another aspect, simulation and/or assistance information can include:

information representing and/or indicating a spacing of two plates of the ligament tensor given by the orientation of a pinion, information representing and/or indicating an intra-prosthetic spacing, information representing and/or indicating an orientation of an upper plate of the ligament sensor, so as to assist in checking whether the distal femur and proximal tibial cuts are indeed parallel to each other, information representing and/or indicating a height of posterior condyle resections, information representing and/or indicating a size of an insert to be implanted, and/or information representing and/or indicating an internal orientation of the femur, so as to assist in placement of a 4-in-1 cutting guide

In another aspect, a program for controlling the system can be stored on a non-transitory computer-readable data storage medium, such as a memory of the system.

Advantageously, the augmented reality display device is a mobile device, such as smart glasses or a mixed reality headset, intended to be worn or carried by the user of the system, typically the surgeon.

### BRIEF DESCRIPTION OF THE DRAWINGS

Further aspects of the invention will be described in reference to the appended drawings which illustrate non-limiting exemplary embodiments, and among which:

FIGS. 1-7 illustrates selected stages of a knee replacement operation:

FIG. 1 is a schematic perspective view of the knee of a human patient, showing the standard incision and the mini-invasive incision.

FIG. 2 is a schematic front view of the knee after incision and installation of bone references on the tibia and on the femur.

FIG. 3 is a schematic view of an augmented reality system with AR goggles worn by a user and an illustration of a field of view of the knee operating scene whose representation is to be displayed by the system.

FIG. 4 is a schematic front view of the knee after positioning of an articulated bone cutting guide on the tibia.

FIG. 5 is a schematic representation of a field of view displayed by an augmented reality system, including a perspective view of the knee and the bone references to which is superposed a 3D model of an articulated bone cutting guide for installation on the tibia.

FIG. 6 is a schematic perspective view of the knee after installation of an articulated bone cutting guide on the femur.

FIG. 7 is a schematic representation of a field of view displayed by an augmented reality system, including a perspective view of the knee and the bone references to which is superposed a 3D model of an articulated bone cutting guide for installation on the femur.

### DETAILED DESCRIPTION OF PARTICULAR EMBODIMENTS

For non-limiting illustration of the invention, exemplary embodiments of the invention will be described in the context of a particular application to knee surgery.

### Example 1: Discussion of Selected Stages of a Knee Replacement Operation Protocol

1. Incision

An incision is performed along the inner wall of the patella, and can be standard incision or less invasive, such as of the mini sub-vastus type, or MIS (“minimal invasive surgery”) incision.

Other skin incisions are not required for use of the navigation system. Pins needed for attachment of the aiming and cutting instruments are all positioned in the main incision in the knee.

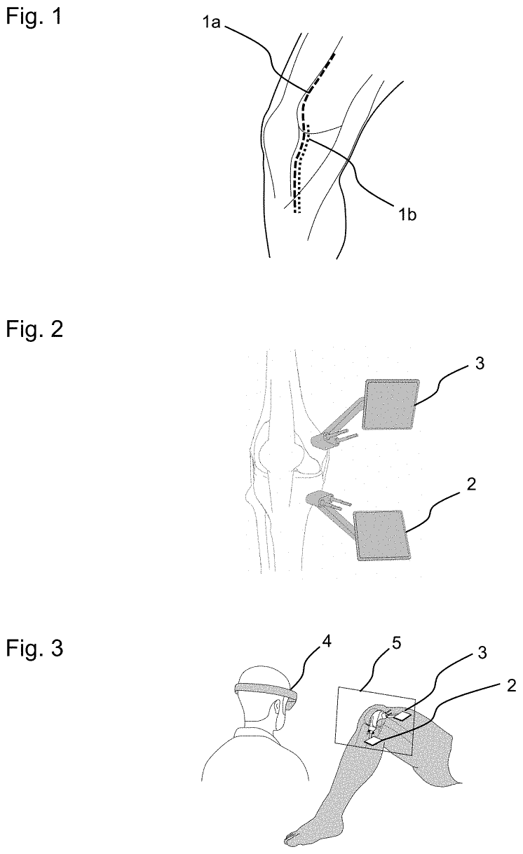

FIG. 1 is a schematic perspective view of the knee of a human patient, showing the typical standard incision 1a, or alternatively, mini-invasive incision 1b.

2. Installing MIS Bone References

The installation of the MIS bone references is carried out using a pre-positioning system allowing an initial orientation of the cutting planes, authorizing the use of a MIS articulated cutting guide with reduced mobility. The pre-positioning system can ensure that the references do not conflict with other instruments. The pre-positioning can be carried out mechanically, or with the assistance of an augmented reality system.

a. Mechanical Pre Positioning

Mechanical pre-positioning is performed using simple mechanical instruments fitted with clear geometric markers that must be positioned in relation to clearly visible anatomical landmarks. Typically, for the tibia, the bone reference pre-positioning instrument is installed according to the top of the tibial spines and the bottom of the tibial plateaus, while for the femur, it is installed according to the surface of the distal condyles and Whiteside's line.

b. Computer-Assisted Pre Positioning

The computer-assisted pre-positioning is carried out essentially through the visualization of 3D objects in augmented reality. During manipulation of a bone reference before installing it on the patient, the surgeon can visualize in real time a simplified 3D model of the bone which is visualized in a position “attached” to the instrument, i.e., related or linked to the position of the instrument, whatever the position of the instrument in space. This way, this pre-positioning can be carried out by superimposing the virtual 3D model of the bone on the real bone of the patient. The virtual 3D model of the bone is, for example, in the form of a hologram or hologram-like representation, or in the form of a semi-transparent rendering. It is possible to guide the surgeon with visual indications according to the approximate data obtained by the perception sensor or sensors. For example, the at least one sensor collects successive clouds of points corresponding to visible articular surfaces and to the overall shape of the leg of the patient, which are stored in a memory, and on the basis of which a processor of the system calculates the distance between the current position of the instruments and a predicted or expected future position.

FIG. 2 is a schematic front view of the knee after incision and installation of bone references 2, 3 on the tibia and on the femur, respectively

3. Acquisition of Mechanical Axes and Geometric References

The acquisition of mechanical axes and geometric references involves defining a minimalist digital model for the tibia and the femur, which can consist in a mechanical axis and an antero-posterior orientation for each bone. This very simple model defines a geometric reference for each bone and allows essential measurements for a knee prosthesis placement: the dynamic HKA (hip-knee-ankle) angle, joint laxity, and the orientation of the cutting planes in real time when the cutting guide is installed. Of course, if desired, additional data can also be included in the digital model.

When the minimalist model of a bone is determined by data acquisition on the patient, the system displays this model, i.e., inter alia or essentially only the axial line and points symbolizing anatomical reference landmarks. This displaying is superposed to the display of the actual anatomy of the patient, thus offering an augmented reality display mode. This display remains superposed to the display of the patient's leg even when the leg is manipulated by the surgeon, provided the bone references are seen or predictable by the perception device of the augmented reality platform, at least a portion of the time. Thus, the virtual models remain attached to the references, i.e., in positions related to the positions of the bone references.

a. Femur Axis and Referential

The femoral mechanical axis is defined as the segment connecting the entry point of the femoral medullary canal (KF point) to the center of the femoral head (H point). The KF point is acquired during the mechanical pre-positioning of the bone reference, and can be registered using a navigated pointer, i.e., a rigid instrument which is usually very simple, having a point used to reach or probe the references and bone surfaces, whose position data is known to the system.

The H point, which is the center of the hip, is acquired by mathematical deduction, typically by a pivot algorithm, from a set of positions of the patient's leg manipulated by the surgeon in a circular motion. This operation requires the presence of a reference fixed in relation to the patient's pelvis, which can be obtained physically by rigidly attaching a reference marker to the surgical operating table, or numerically by using SLAM (simultaneous localization and mapping) technology, considering the patient's pelvis as being immobile in the operating room space.

The antero-posterior orientation is also obtained during the mechanical pre-positioning of the bone reference, and can also be acquired using the navigated pointer. It is defined as the direction of Whiteside's line, or alternatively as the direction perpendicular to the plane passing through the two posterior condyles. Whatever definition is chosen, this orientation is only used to define the geometric reference frame according to which the orientation values of cutting planes and instruments will be calculated by projection. The difference in orientation between Whiteside's line and the line normal to the plane of the posterior condyles is such that with an accuracy of one degree, the calculated orientation values are the same regardless of the anatomical reference chosen.

b. Tibia Axis and Referential

The tibial mechanical axis is defined as the segment connecting the entry point of the tibial medullary canal (K_{T} point) to the center of the ankle (A point). The K_{T} point is acquired during the mechanical pre-positioning of the bone reference, and can be registered using the navigated pointer. The A point is defined as the center of the segment connecting the malleolus, and is obtained by registering the apex of each malleolus with the navigated pointer.

The antero-posterior orientation is also obtained during mechanical pre-positioning of the bone reference, or it can also be acquired using the navigated pointer. It is defined as the direction of the line connecting the insertion point of the posterior cruciate ligament and the medial third of the anterior tibial tuberosity. As for the femur, an imprecision of this orientation has normally little impact on the orientation values calculated by projection.

FIG. 3 is a schematic view of an augmented reality (AR) system with AR goggles 4 worn by a user and a symbolic illustration of a field of view 5 of the knee operating scene whose representation is to be displayed by the system.

4. Acquisition of Articular Surfaces

The acquisition of articular surfaces makes it possible to bring details to the digital model of the tibia and the femur. A 3D mesh of the articular surface of each bone is obtained, and calculations and simulations are performed to assist the optimal positioning of the prosthetic components.

The system can display these augmented reality 3D meshes to the surgeon directly on the patient's knee joint surfaces. It thus indicates to the surgeon which articular surfaces are already acquired and which are not or require more details. When the virtual model of the patient's knee is sufficiently detailed, the augmented reality display allows the surgeon to visualize the articulation through the skin of the patient in augmented reality. Indeed, since the virtual models remain digitally attached to the bone references rigidly attached on the patient's femur and tibia, they can be displayed at any stage of the protocol, superimposed on the patient's knee whether the articular surfaces are visible or hidden under the skin.

Various procedures are available for this purpose, alternatively or in combination, among which are the procedures described below.

a. Acquisition of Point Articular Landmarks

This articular surface acquisition mode is usually the simplest. It consists in registering only a few 3D points using the pointer, allowing calculations of relevant sizes and distances.

For the femur, the user can point to the apex of each condyle, i.e., the most distal point of the articular surface internally and externally. This makes it possible to quantify the thickness of the distal femoral cut, during adjustment of the cutting plane, and after verification using the control plate. Alternatively, or additionally, the user can also capture the most posterior point of each condyle for rotation and space in flexion calculations, as well as the insertion points of the lateral ligaments on the epicondyles and anterior cortex, to allow measurement of the size of most appropriate femoral prosthetic component for the patient.

For the tibia, the user can point to the bottom of each plateau, i.e., the deepest point of the tibial articular surface internally and externally. This makes it possible to quantify the thickness of the proximal tibial cut, during adjustment of the cutting plane, and after verification using the control plate. Alternatively, or additionally, the user can also acquire the most lateral and medial points of the tibial epiphysis, to allow measurement of the most appropriate tibial prosthetic component size for the patient.

b. Acquisition of Surfaces by Manual Scanning

This acquisition mode is often used in navigation systems. It involves manually scanning the articular surfaces of the femur and the tibia using the pointer in order to collect 3D point clouds allowing a detailed digital reconstruction of the knee joint, by smoothing out point clouds, or deformation of statistical models (“bone morphing”).

For the femur, the user must scan the articular surfaces according to the following zones: internal distal condyle, external distal condyle, internal epicondyle, external epicondyle, internal posterior condyle, external posterior condyle, trochlea and anterior cortex.

For the tibia, the user must scan the articular surfaces according to the following zones: internal plateau, external plateau, circumference of the epiphysis.

c. Acquisition of Surfaces by Advanced Digital Perception

This mode of acquisition uses the perception sensor or sensors of the augmented reality platform to directly digitize the articular surfaces of the knee at the start of the intervention, typically using depth sensor(s) and/or high-definition camera(s). The user simply “films” the articular surface in as many directions as possible, thus allowing the various sensors to acquire 3D point clouds without using the pointer. These point clouds are then used for the reconstruction of the articular surfaces, in the same way as when they are acquired using the pointer.

d. Acquisition of Surfaces by Registration of Scanner/MRI Data

This mode of acquisition involves the production of DICOM (digital imaging and communications in medicine) data, such as scanner and/or MRI (magnetic resonance imaging) images, of the patient's knee prior to the intervention, as well as the segmentation of this data to obtain a detailed 3D model of the joint.

During the intervention, once the knee has been incised and the bone references have been installed, the user can proceed with the registration of the DICOM data thanks to the acquisition of a few clearly identifiable anatomical landmarks on the patient's femur and Tibia. These anatomical landmarks can simply be those detailed in procedure a. above. The user can visualize in real time the DICOM model superimposed on the real anatomy of the patient, and thus visually check the correct registration of this first.

Once the DICOM data on the tibia and the femur has been registered, the system can perform very precise calculations of distances and orientations, as well as geometric and dynamic simulations allowing the display of relevant information for the surgeon and the optimization the positioning of the prosthetic components.

5. Dynamic Acquisition of Joint Kinematics Before Fitting the Prosthesis

These acquisitions are optional because the prosthesis can be installed precisely on the patient's knee without having to resort to it. Above all, they make it possible to offer the surgeon advanced functionalities allowing the surgeon a better understanding of the kinematics of the operated knee and detailed simulation possibilities in order to be able to test prosthetic positioning configurations before performing the bone cuts.

a. Ligament Laxity

The dynamic acquisition of joint kinematics typically involves carrying out two complete strokes of the patient's knee, from total extension to maximum flexion, in order to capture for any flexion angle value, the ligament laxity expressed as the value of the HKA angle in the frontal plane. Since it is desired to measure the entire range of laxity, the user must perform a full stroke by putting the internal ligament under tension, and a second stroke, this time putting the outer ligament under tension. This acquisition requires the creation of at least minimalist digital bone models (oriented mechanical axes), but preferably, detailed models are used for more complete and relevant measurements, such as the measurements of the medial and/or lateral intra-articular spaces.

The results of this acquisition are presented in the form of a laxity graph, advantageously displayed in a floating window in augmented reality, with the ordinate being the knee flexion angle value and the abscissa being the HKA angle value describing the laxity, or even, if the model of the knee is sufficiently detailed, the value of the intra-articular spaces. The results are divided into two curves, internal laxity and external laxity, which, on the graph, may exhibit a kind of deformation or “bubble” on the surface, more or less significant depending on the symptoms presented by the knee.

The user can perform this acquisition just after the incision at the start of the operation, to measure the laxity of the diseased knee, then again after placement of trial implants or permanent implants, in order to check the stability of the prosthetic knee. An improvement in the stability of the knee thanks to the implantation of the prosthesis is reflected, on the laxity graph, by a significant reduction in the surface of the “bubble” described by the two HKA value curves.

b. Patella Kinematic

This operation consists in acquiring, at the beginning of the intervention, a measurement of the trajectory and the position of the patella during the cycle of flexion-extension of the operated knee. Technically, this operation requires the attachment of a navigation marker to the patient's patella, or the development of an algorithm that can infer the position and orientation of the patella through the skin based on a cloud of 3D points acquired by a depth sensor of the augmented reality platform. This acquisition will preferably be carried out before the incision, in order to keep intact all the soft tissues and their role in the kinematics of the joint.

The result of this acquisition can be visualized in the form of a 3D curve displayed superimposed on the patient's knee, or on a virtual representation of the patient's joint in a floating window. The positions and orientation acquired are used to feed the biomechanical simulation of the implant positioning (see, e.g., point 13 below).

6. Configuration of the Positioning of the Tibial Implant

The user can visualize in real time a 3D model of the implant superimposed in augmented reality on the real knee of the patient. As the patient's tibia and femur are localized in space, the virtual implant models appear to be “attached” to the patient's bones, allowing the user to dynamically simulate interactions between prosthetic components and potential conflicts before making any cuts.

a. Manual Positioning

The navigation software interface allows the user to manually adjust each of the degrees of freedom of the positioning of the implants. The user can freely define the position of the implant by defining, notably, one or more of the following: its size, its orientation in varus/valgus, its orientation in flexion-extension, the thickness of the tibial cut, its medio-lateral positioning, and its antero-posterior positioning.

b. Anatomical Positioning

If the user has made at least occasional acquisitions of anatomical landmarks on the patient's knee, or even acquisitions of articular surfaces, or at best a DICOM data adjustment, the navigation software is capable of suggesting a positioning of the prosthetic components reproducing the patient's existing joint surfaces as well as possible. The purpose of this positioning of the implant is to modify the anatomy of the operated knee as little as possible, preserving the spaces and anatomical angles measured, and following a philosophy of fitting a knee prosthesis qualified as “independent cuts”, because the positioning of the femoral component does not take into account the position of the tibial component, and vice versa.

c. Positioning in Mechanical Alignment

Whatever the level of detail of the model defined for the operated knee, the system can suggest to the user a positioning of the prosthetic components according to a mechanical alignment, that is to say based on the mechanical parameters of the knee. This alignment essentially consists of orienting the prosthetic components on the mechanical axes of the Tibia and the Femur, adapting this orientation if necessary to the specificities of each implant model. In the case of the tibial component, this amounts to defining a neutral valgus and a posterior slope usually between 3 and 7 degrees.

d. Positioning According to Dynamic Acquisitions (Inverse Kinematics)

If the user has performed alignment and space measurements using the flexion and extension tensor, the navigation software can propose a positioning of the prosthetic components making it possible to reproduce exactly the configurations of the leg measured, once the prosthetic components have been placed. This is the installation philosophy qualified as “dependent cuts”, because the positioning of the prosthetic components is planned as a whole, as a system of articulated components, which makes it possible to control the stability of the knee, the kinematics of the different components, and the ligament tension over the entire flexion range of the joint. It is a functional approach to the positioning of prosthetic components, also known as the inverse kinematics approach, because it does not seek to impose a new kinematics on the operated knee, but rather to respect and recover the natural kinematics of the joint.

7. Installation of the Articulated Cutting Guide on the Tibia

The articulated cutting guide is installed on the tibia by fixing the articulated cutting guide on the tibia of the patient with surgical pins or screws. Thanks to the pre-positioning of the instruments, the cutting plane defined by the slot of the cutting guide is located in the expected or predicted zone, which makes it possible to offer a compact sighting/cutting instrument with a low adjustment range.

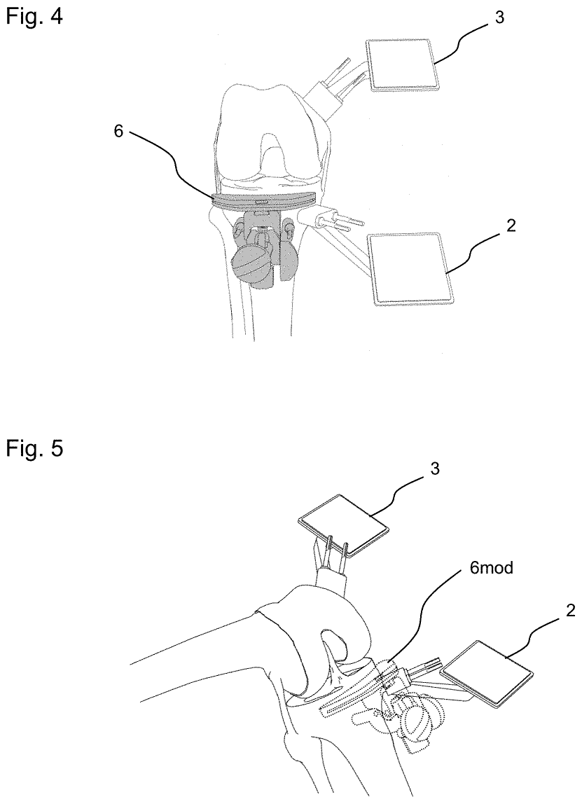

FIG. 4 is a schematic front view of the knee after positioning of an articulated bone cutting guide 6 on the tibia.

FIG. 5 is a schematic representation of a field of view displayed by an augmented reality system, including a perspective view of the knee and the bone references to which is superposed an articulated bone cutting guide 3D model 6mod for installation on the tibia.

8. Adjustment of the Proximal Tibial Cutting Plane

As soon as the articulated cutting guide is attached to the bone, the system displays for the user a virtual view of the current cutting plane, materialized by the cutting guide slot, as well as the orientation values of the cutting plane in degrees, e.g., varus/valgus and flexion/extension, and/or resection thickness, typically in millimeters. The user can choose the reference used for the calculation of the level of resection: internal plateau or external plateau.

Advantageously, the articulated cutting guide is equipped with at least three adjustment functions allowing the cutting plane to be configured manually: a wheel for the adjustment of the flexion/extension angle, another for the adjustment of the varus/valgus angle, and a third for adjusting the level of resection. The user can observe simultaneously or quasi-simultaneously on the augmented reality display the updating of the virtual cutting plane and orientation values when manipulating the various settings on the instrument.

Advantageously, the user can choose between a “real-time” cutting plane adjustment mode, i.e., in which the orientation values displayed allow the surgeon to reach the desired orientation, for example, according to the practice of the surgeon or the recommendations of the operating technique, and a “planned” adjustment mode in which the user seeks to achieve an orientation of the cutting plane previously defined in the system, manually and/or automatically thanks to the various simulation tools and positioning aid for the available components. The system can then display to the user the “current” section plane of the instrument in place at the same time as the “planned” section plane, in which case the adjustment of the section consists, among other things, in making these two planes coincide. thanks to the adjustments of the articulated cutting guide.

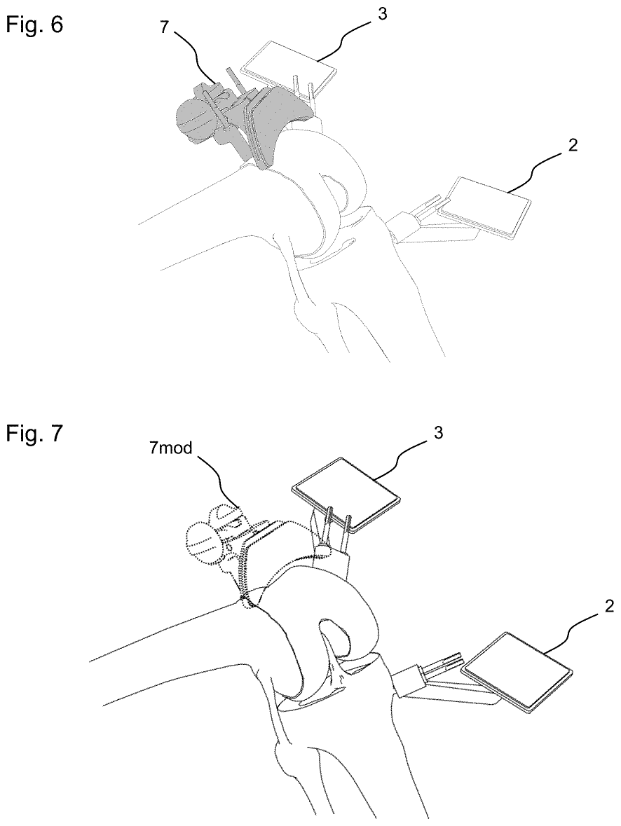

FIG. 6 is a schematic perspective view of the knee after installation of an articulated bone cutting guide 7 on the femur.

FIG. 7 is a schematic representation of a field of view displayed by an augmented reality system, including a perspective view of the knee and the bone references to which is superposed an articulated bone cutting guide 3D model 7mod for installation on the femur.

9. Tibial Cut

The tibial cut is made through the hinged cutting guide slot with a standard oscillating saw blade, for example, 1.27 mm. The cut can be made directly after the adjustment of the cutting plane has been validated, but it is typically recommended to stiffen the fixation of the cutting guide to the patient's bone using pins or screws with a diameter of 3.2 mm, for example, after it has been translated in contact with the cortical bone.

10. Verification of the Tibial Cut

Once the proximal tibial cut has been made and the portion of resected bone removed from the joint, the user can check the orientation of the cut made with the instrument called a control plate. This is provided with a flat surface that the user must bring into contact with the bone section created by the cut in order to acquire its orientation. The system can then display to the user either the orientation of the plane in the geometric reference of the bone, or the angular values of deviation with respect to the initially planned cut.

11. Installation of the Ligament Tensor

The purpose of the ligament tensor is to tension the lateral ligaments of the patient's knee, in order to perform functional measurements on the patient's leg. In extension, the use of the tensor makes it possible to check the alignment of the leg, e.g., the angle HKA, induced by the soft tissues, independently of the future positioning of the implant. In flexion, the use of the tensor makes it possible to quantify the femoral rotation, that is to say the angle between the plane of the posterior femoral condyles and the plane of the tibial cut.

In order for the measurements to best reflect dynamic operating positions of the knee, and in order to avoid bias induced by wear of the joint or osteophytes applying undesirable stresses on the ligaments, the tensor is preferably used after the osteophytes have been removed and the ligaments have been freed of any abnormal stress.

The tensor is inserted into the knee joint, and is equipped with a first plate which must rest flat on the tibial cut. Its design allows its installation in the knee and taking measurements with the patella in place, i.e. with the knee incision almost closed and the patella in its normal operating position, in the femoral trochlea. It is fitted with a control, such as a screw, rack, trigger, etc., which separates the mobile component, i.e., typically a second plate, resting under the femoral condyles, from the static component resting on the tibial cut, i.e., the first plate. The user manipulates this control until the desired tension is obtained, which can be quantified simply by the physical difficulty of creating the tension, or by using the instrument's built-in tension quantization function, for example, compression of a spring, torque screwdriver, etc.

The navigation system, through the sensors of the mobile device, is able to read the information of the tensor during its use, to memorize it, and to display it in the interface of the software, and to integrate it into the simulation tools and aid in the positioning of prosthetic components.

The tensor can be used for verification and quantification. In particular, when the tensor is equipped with a function for quantifying the applied tension, this allows the surgeon to apply the same tension in the knee in extension as in the knee in flexion. This allows the surgeon to avoid bias in the measurements of spaces and femoral rotation with a difference in tension applied between flexion and extension.

In extension, the navigation system will read the spacing of the two plates given by the orientation of the pinion, which makes it possible to know the intra-prosthetic spacing. At the same time, reading the orientation of the upper plate will be used to check whether the distal femur and proximal tibial cuts are indeed parallel to each other.

In flexion, the spacing of the plates makes it possible to determine the intra-prosthetic spacing in this position. With the various data potentially recorded, the navigation system can di splay the height of posterior condyle resections and the size of the insert to be implanted, typically a polyethylene insert. Reading the orientation of the upper plate with the patella in place provides the internal orientation of the femur. This value can be used for placement of a 4-in-1 cutting guide used to perform most of the femoral bone cuts.

12. Measurements of Joint Angles and Spaces Under Ligament Tension

Using the tensor in conjunction with the rest of the augmented reality navigation system has several advantages:
The computer vision system can directly read the measurements on the tensor and store them, using specific graduations and a 3D location marker unique to the instrument. This allows the surgeon to avoid potential errors when choosing certain prosthesis orientation parameters, and to keep track of these measurements in the surgery report.
The navigation system can record the respective positions and orientation of the tibia and the femur, and consequently extrapolate the positions and orientations of one relative to the other under the constraint of the tensor, which can constitute data of relevant input for positioning aid of prosthetic components, especially the femoral component.

The tensor can be used with the augmented reality system so as to acquire data in extension and/or in flexion.

a. In Extension

The tensor can be used before the distal femoral cut, in which case it is essentially used to put the knee in tension in order to digitally measure the functional HKA angle (induced only by the ligaments and soft tissues), or after the distal cut in which case it is mainly used to check the parallelism between the distal femoral and proximal tibial cuts, as well as to quantify the knee prosthesis polyethylene insert size required for the patient's knee.

b. In Flexion

The main function of the tensor used in flexion is to tension the lateral ligaments, which causes rotation (most often internal) of the femur and thus makes it possible to measure its rotation value in flexion, either directly on the instrument, or by digital measurement by computer vision. This value is memorized by the navigation software in order to be taken into account in the positioning of the implant on the femur.

The tensor used in flexion also makes it possible to quantify the intra-prosthetic space in flexion, which makes it possible to determine (or confirm) the appropriate polyethylene insert size required for the patient's knee.

13. Configuration of the Positioning of the Femoral Implant

As for the tibia, the user can visualize in real time a 3D model of the implant superimposed in augmented reality on the real knee of the patient and dynamically simulate the interactions between the components. Several options are available to define the positioning of the femoral implant, which may be used alternatively, successively, or in combination of two or three or more: manual positioning, anatomical positioning, mechanical alignment positioning, dynamic acquisition positioning (inverse kinematics), digital twin simulation.

a. Manual Positioning

As for the tibia, the user can freely define the position of the implant by defining: its size, its orientation in varus/valgus, its orientation in flexion extension, the distal cut thickness, its medio-lateral positioning and its anteroposterior positioning.

b. Anatomical Positioning

This is the same procedure as for the tibia.

c. Positioning in Mechanical Alignment

This is the same procedure as for the tibia.

d. Positioning According to Dynamic Acquisitions (Inverse Kinematics)

If the user has performed alignment and space measurements using the flexion and extension tensor, the navigation software can propose a positioning of the prosthetic components making it possible to reproduce exactly the configurations of the leg measured, once that the prosthetic components have been placed. This is the installation philosophy qualified as “dependent cuts”, because the positioning of the prosthetic components is planned as a whole, as a system of articulated components, which makes it possible to control the stability of the knee, the kinematics of the different components and the ligament tension over the entire flexion range of the joint. It is a functional approach to the positioning of prosthetic components, also known as the inverse kinematics approach, because it does not seek to impose a new kinematics on the operated knee, but rather to respect and recover the natural kinematics of the joint.

e. Positioning According to Digital Twin Simulations

This approach to the positioning of prosthetic components is advantageously complementary to previous approaches. It is based on the possibility of simulating the complete kinematics of the joint, taking into account the ligament tensions, for any configuration of the prosthetic components. It requires an appropriately detailed model of the joint obtained by direct acquisition of the articular surfaces, or registration of DICOM data. The user can thus simulate in real time the consequences in terms of conflicts and tension, of a given planning of implant positions, whether carried out manually, by geometric or functional positioning. The simulation also allows the navigation software to directly suggest a positioning of the prosthetic components optimizing a certain parameter, such as knee stability, or ligament tension. The user simply specifies objectives, constraints and/or priority criteria and the software offers a positioning of the prosthetic components that optimizes them.

Typically, the approach will involve: digitization/adjustment of the surface, acquisition of the insertion of the ligaments, trajectory and conflict prediction, navigation/suggestion of the patellar cut for optimized performance.

14. Installation of the Articulated Cutting Guide on the Femur

The articulated cutting guide used for the tibial cut can also be used for the distal femoral cut. The part comprising the cutting slot is modular and removable from the rest of the instrument. There are several versions to adapt to different approaches and surgical techniques: symmetrical (for the femur), partial right or left (for the tibia) or right or left lateralized (for the MIS routes).

15. Adjusting the Distal Femoral Cutting Plane

This is the same procedure as for the proximal tibia cut.

16. Distal Femoral Cut

This is the same procedure as for the proximal tibia cut.

17. Adjustments for the Other Femur Cuts

The adjustments for the other femur cuts can be made using a drilling guide for the 4-in-1 cutting guide, and/or using a navigated adjustment instrument in augmented reality, for example.

a. With Drilling Guide for 4-In-1 Cutting Guide

When the distal femoral cut has been made, the four remaining cuts to install the femoral implant are made using a cutting guide called “4-in-1” because it includes the four cutting slots corresponding to the four cuts to be made for the size of the chosen implant. This cutting guide is installed flat on the distal femoral cut and is attached to the operated-on femur using at least two pointed pins or spikes. The adjustment of the 4 remaining femoral cuts using the system therefore consists of assisting the surgeon in making the two holes corresponding to the 4-in-1 cutting guide appropriate to the chosen component. This operation can be carried out using a drilling guide navigated by the system, essentially consisting of a metal block having two holes and equipped with a navigation marker. The surgeon manipulates this guide freehand, placing it flat on the distal cut and being guided by the system using visual aids displayed directly superposed on the patient's joint. The user can view a hologram of the instrument in an attempt to superimpose the real instrument on it, or use symbols and color codes indicating the distance and direction to move the instrument. When the system validates the current position of the instrument, the user can drill the two guide holes of the 4-in-1 cutting guide in the distal face of the femur.

b. Using a Navigated Adjustable Instrument

This approach is quite similar to that presented in the previous paragraph, it differs by the instrument used, which in this case is not navigated by the user freehand, but is manipulated while it is rigidly attached to the patient's femur throughout the adjustment phase. Mechanisms allowing the user to independently set each cutting plane using the system and perform the remaining four femoral cuts one after the other. The system assists the user throughout this adjustment phase using visual information displayed superimposed on the patient's femur.

c. Control of Femoral Cuts

Same procedure as for the tibia, with the use of the navigated control plate.

18. Trial Implants and Control Kinematics Analysis

When all the bone cuts have been made, the surgeon can install temporary implants often called trial implants, with which the surgeon can control the kinematics and stability of the joint before the placement of the definitive prosthetic components. During this control phase, the user can repeat a dynamic acquisition of joint kinematics, for example, as that presented above in paragraph 5, in order to observe the improvement in joint laxity following the placement of the trial implants.

If the results observed during this phase seem abnormal, the surgeon can return to any stage of the system's operating protocol to modify parameters and/or redo acquisitions on the patient as long as he has not removed the bony references attached to the tibia and the femur. The surgeon can also modify the bone cuts by replacing the articulated cutting guide in its initial fixing holes.

19. End of Protocol

When the surgeon is satisfied with the last checks, the surgeon closes the operating protocol of the system by validating the end of the intervention. The system can then produce a surgery report that the user can consult on the platform or receive as a document by email. This report contains all the data that characterizes the intervention and offers a summary of the orientation parameters of the implants and the bone cuts performed.

### ADDITIONAL COMMENTS

Various technological blocks can be used in the practice of embodiments of the methods according to the invention, which may include non-limitatively, alone or in any successions or combinations of two or more, among the following:

QR codes

ArUco markers

SLAM algorithms

Advanced perception sensors, typically visual perception sensors, optical sensors (StereoVision, LIDAR, TOF sensor, etc.)

Installation estimation based on a model (typically a 3D model) of objects or parts in the operating scene

Deep learning

Waveguide display

See-through HMD (head mounted display)

Augmented reality makes it possible to display virtual information in the operating scene directly in the surgeon's field of vision, either superimposed on the reality perceived by the surgeon (“mixed reality” vision) or superimposed on a video stream displayed in the surgeon's field of vision (“augmented reality” vision). The augmented reality visualization can provide additional safety to the surgeon because the system can display critical information directly in the operating scene, and/or present the surgeon with virtual models and/or control elements displayed directly in “transparency” through the patient's soft tissues or instruments that would normally be hidden. For example, the system can integrate an augmented reality headset and/or augmented reality glasses, such as by Vuzix, Rochester, N.Y., for example, a model M400. The augmented reality display can be a single 2D image view with superposed information, or an actual view by the eye of the user, with superposed information provided by the device, or even a virtual simulation of an actual view with superposed information, or any combination of these augmented reality displays. The virtual simulation of an actual view can be created based on an actual view, on a model, such as a 2D or 3D model, of the actual view, from a predefined model, such as a 2D or 3D model, or any combination of these simulations. A portion of an actual view or of an image of an actual view may be excluded or occluded in favor of the augmented reality information, such as by framing and/or covering.

The above disclosures, embodiments and examples are nonlimitative and for illustrative purposes only.

1. A method of fitting a knee prosthesis in a knee of a patient, the method comprising:
displaying, by a visual display device of a mobile augmented reality navigation system worn or carried by a user performing or assisting at least one navigation-assisted stage of the method, at least one visual element superposed on a view of at least a portion of a surgical scene of the fitting of the knee prosthesis,
the at least one visual element being selected from the group consisting of a 3D model of at least one portion of the knee of the patient, a 3D model of another component of the surgical scene, information relative to the at least one portion of the knee of the patient, and information relative to the other component of the surgical scene.

2. The method of claim 1, wherein the at least one navigation-assisted stage of the method includes performing a manual action by the user or by a person assisted by the user, the method comprising:
initiating the performance of the manual action, and
modifying the performance of the manual action taking into account the at least one visual element.

3. The method of claim 1, wherein the at least one navigation-assisted stage of the method includes at least one action selected from the group consisting of:
performing an incision in the knee,
installing at least two bone references,
acquiring data on mechanical axes and geometric references related to a knee joint of the patient,
acquiring data on articular surfaces of the knee joint,
dynamically acquiring data on kinematics of the knee joint,
installing an articulated cutting guide on a tibia of the patient,
adjusting a position of a proximal tibial cutting plane,
performing a tibial cut along the tibial cutting plane,
verifying the tibial cut,
installing a ligament tensor,
measuring joint spaces under ligament tension,
determining a configuration and a placement of a femoral implant,
installing an articulated cutting guide on a femur of the patient,
adjusting a position of a distal femoral cutting plane,
performing a distal femoral cut along the distal femoral cutting plane, and
fitting at least part of a knee prosthesis.

4. The method of claim 3, wherein the at least one navigation-assisted stage of the method includes at least two actions selected from the group.

5. The method of claim 3, wherein the at least one navigation-assisted stage of the method includes at least one action selected from the group consisting of:
installing a ligament tensor,
measuring joint spaces under ligament tension,
determining a configuration and a placement of a femoral implant.

6. The method of claim 5, wherein the at least one navigation-assisted stage of the method includes:
successively acquiring, by a perception sensor of the navigation system, values relative to a configuration of the ligament tensor in a plurality of relative positions of the femur and the tibia,
successively displaying, by the visual display device, information relative to the configuration of the ligament tensor in each of the plurality of relative positions, and
successively displaying, by the visual display device, simulation and/or assistance information relative to the configuration of the ligament tensor and/or other components of the knee prosthesis.

7. The method of claim 1, wherein the at least one navigation-assisted stage of the method comprises:
successively displaying, by the visual display device, a position and/or an orientation of the at least one portion of the knee of the patient or other component of the surgical scene, in each of a plurality of relative positions of the at least one portion of the knee of the patient or other component of the surgical scene.

8. The method of claim 7, wherein the at least one navigation-assisted stage of the method further comprises:
successively displaying, by the visual display device, simulation and/or assistance information relative to the positioning in the surgical scene of at least one portion of a surgical instrument.

9. The method of claim 7, wherein the at least one navigation-assisted stage of the method further comprises:
successively displaying, by the visual display device, simulation and/or assistance information relative to the positioning in the surgical scene of at least one portion of the knee prosthesis.

10. The method of claim 1, wherein the at least one navigation-assisted stage of the method comprises:
displaying, by the visual display device, simulation and/or assistance information relative to the positioning of at least one portion of a surgical instrument in the surgical scene.

11. The method of claim 1, wherein the at least one navigation-assisted stage of the method comprises:
displaying, by the visual display device, simulation and/or assistance information relative to the positioning of at least one portion of the knee prosthesis in the surgical scene.

12. The method of claim 1, wherein the at least one navigation-assisted stage of the method includes fitting at least part of the knee prosthesis.

13. The method of claim 1, comprising:
storing, in a memory included in or connected to the navigation system, successive visual elements relative to positions and/or an orientations of the at least one portion of the knee of the patient or of the other component of the surgical scene.

14. The method of claim 13, comprising:
displaying, by the visual display device, the successive visual elements stored in the memory.

15. The method of claim 1, wherein at least one perception sensor is included in or connected to the navigation system.

16. The method of claim 15, wherein the at least one perception sensor includes at least one camera.

17. The method of claim 1, wherein the mobile augmented reality navigation system includes augmented reality glasses and/or an augmented reality headset comprising the visual display device.

18. The method of claim 1, wherein the visual display device shows at least one selected form the group consisting of
information representing and/or indicating a spacing of two plates of the ligament tensor given by the orientation of a pinion,
information representing and/or indicating an intra-prosthetic spacing,
information representing and/or indicating an orientation of an upper plate of the ligament sensor, so as to assist in checking whether the distal femur and proximal tibial cuts are indeed parallel to each other,
information representing and/or indicating a height of posterior condyle resections,
information representing and/or indicating a size of an insert to be implanted,
information representing and/or indicating an internal orientation of the femur, so as to assist in placement of a 4-in-1 cutting guide.

19. A mobile augmented reality navigation system for assistance in fitting a knee prosthesis, the navigation system comprising a visual display device, management software and at least one perception sensor, wherein the system is adapted to implement a method of fitting a knee prosthesis in a knee of a patient, the method comprising:
displaying, by a visual display device of the mobile augmented reality navigation system worn or carried by a user performing at least one navigation-assisted portion of the method, at least one visual element superposed on a view of at least a portion of a surgical scene of the fitting of the knee prosthesis,
the at least one visual element being selected from the group consisting of a 3D model of at least one portion of the knee of the patient, a 3D model of another component of the surgical scene, information relative to the at least one portion of the knee of the patient, and information relative to the other component of the surgical scene.

20. A non-transitory computer-readable data storage medium on which is stored program comprising navigation instructions for a mobile augmented reality navigation system for assistance in fitting a knee prosthesis, the navigation system comprising a visual display device, management software and at least one perception sensor, wherein the program is adapted, when the program is run on a computer, to control the system to implement a method comprising:
displaying, by a visual display device of a mobile augmented reality navigation system worn or carried by a user performing at least one navigation-assisted portion of the method, at least one visual element superposed on a view of at least a portion of a surgical scene of the fitting of the knee prosthesis,
the at least one visual element being selected from the group consisting of a 3D model of at least one portion of the knee of the patient, a 3D model of another component of the surgical scene, information relative to the at least one portion of the knee of the patient, and information relative to the other component of the surgical scene.
